(19) United States
(12) Patent Application Publication
(10) Pub. No.: US 2015/0201969 A1
(43) Pub. Date: Jul. 23, 2015

(54) Skin Exfoliating Device

(71) Applicant: FOOT INNOVATIONS, LLC, Belmont, N (US)

(72) Inventors: Troy Dugo, Belmont, NC (US); David Fussell, Cherryville, NC (US)

(73) Assignee: FOOT INNOVATIONS, LLC, Belmont, NC (US)

(21) Appl. No.: 14/588,675

(22) Filed: Jan. 2, 2015

Related U.S. Application Data
(60) Provisional application No. 61/923,878, filed on Jan. 6, 2014.

(51) Int. Cl. A61B 17/54

(52) U.S. Cl. CPC A61B 17/54

(57) ABSTRACT

The present invention is a water-resistant, battery-powered, motorized, spinning, hands-free skin exfoliating device comprising a housing and an abrasive surface. The abrasive surface comprises a body and a plurality of abrasive elements positioned on the exterior face of the body. The body and the plurality of abrasive elements are together formed from a single piece of the same material. The housing contains a power source and a motor. The device is preferably placed on the floor of a shower when in use. When activated, the motor causes the abrasive surface to spin so that the abrasive surface will exfoliate dead skin and calluses from a location on a foot when the location on the foot is placed against the spinning abrasive surface.

### CROSS-REFERENCE TO RELATED APPLICATIONS

The present application claims the benefit of U.S. Provisional Application No. 61/923,878, filed Jan. 6, 2014, which is incorporated by reference herein in its entirety.

### BACKGROUND

Skin exfoliation involves the removal of dead skin cells on the skin's outermost surface. People use skin exfoliation to help maintain healthy, beautiful, and soft skin. Additionally, skin exfoliation can help reduce discomfort associated with calluses and corns which may form on the bottoms of the feet. Skin exfoliation can be achieved through mechanical or chemical means. For example, common forms of mechanical skin exfoliation involve rubbing the skin with pumice, micro-bead scrubs, sponges, loofahs, cloths, or other abrasives. Chemical exfoliant products may contain salicylic acid, glycolic acid, fruit enzymes, citric acid, malic acid, or alpha hydroxyl acid.

Exfoliating the skin from the bottoms of the feet is often performed after soaking feet in water. This softens the hard, dead skin and makes it easier to remove. An ideal environment for wetting the skin is the shower. However, many skin exfoliating devices are not water resistant and can either be damaged by water or harm the user if used in a wet environment like the shower. Therefore, there is a need for a skin exfoliating device that is water resistant.

Exfoliating the skin from the bottoms of the feet is often performed using mechanical means, such as by rubbing the hard, dead skin with an abrasive. This process requires repeated rubbing using moderate pressure. However, many skin exfoliating devices are manual and require a user to expend substantial effort to achieve quality results. This can be difficult for people with disabilities, the elderly, or people that for other reasons lack sufficient strength or stamina. Many powered devices must be plugged in to an AC power source in order to function. This reduces the portability of the device and makes it dangerous to use in a wet environment. Therefore, there is a need for a skin exfoliating device that is battery powered, motorized, and spinning.

Exfoliating the skin from the bottoms of the feet is often performed using hand-held abrasive surfaces. However, it can be difficult for some people to reach the bottoms of their feet, either because of physical limitations (such as injury or inflexibility of the back), lack of proper seating, or lack of physical space to accommodate the body in a seated or bent over position. Therefore, there is a need for a skin exfoliating device that is hands free.

Exfoliating the skin from the bottoms of the feet is often performed using flat abrasive surfaces. However, it can be difficult or uncomfortable for people to exfoliate skin along the convex and concave contours of the bottom of the foot (such as the heel, ball, or arch of the foot) using a flat abrasive surface. Therefore, there is a need for a skin exfoliating device that has a curved shape that is complementary with the contours of the foot.

Exfoliating the skin from the bottoms of the feet is often performed using abrasive surfaces or abrasive elements that are made from a different material than that from which the rest of the device has been made. For example, many abrasive surfaces have abrasive elements made of grains of pumice stone that are glued on to a cloth, paper, or other type of pad. As a result, the abrasive elements may be worn away or break off over time or the entire abrasive surface may peel or tear away from the rest of the device. Other abrasive surfaces are made from textured metal or loofah and fastened to the rest of the device using screws or other types of fasteners. This increases the complexity and cost of manufacturing. In neither case is it possible to utilize common and inexpensive injection molding manufacturing methods to create the entire abrasive surface including the abrasive elements. Therefore, there is a need for a skin exfoliating device with an abrasive surface that is formed from a single piece of the same material used to make substantially the rest of the device that can be manufactured using injection molding methods.

Information relevant to attempts to address these problems can be found in U.S. Pat. Nos. 8,545,516, 6,178,970, 7,270,641, 6,740,052, 8,162,956, 6,142,156, 7,712,474, and 7,278,431. However, each one of these references suffers from one or more of the following disadvantages: the device is not water resistant, the device is not battery powered, the device is not motorized, the device lacks a spinning abrasive surface, the device is not hand held, the device lacks an abrasive surface with a shape that is compatible with the contours of the bottom of a person's foot, or the device lacks an abrasive surface that can be manufactured as a single piece using injection molding manufacturing methods.

For the foregoing reasons, there is a need for a skin exfoliating device for feet that is water resistant, battery powered, motorized, spinning, free standing, compatible with the contours of the bottom of a person's foot, and easy to manufacture.

### SUMMARY

The present invention is directed to a device that satisfies this need for a water-resistant, battery-powered, motorized, spinning, free-standing skin exfoliating device that is compatible with the contours of the bottom of a person's foot and is easy to manufacture. The skin exfoliating device having the features of the present invention comprises a generally cylindrical housing having a base and a top connected to the base. Preferably, the housing is water resistant so that the device can be used in a wet environment like the shower. The device further comprises an on-off switch positioned on the exterior side of the housing that is electrically connected to a power source and a motor, both positioned within the housing. Preferably, to facilitate access to the power source, which may be at least one battery, an access door is located on the base of the housing. The device further comprises an abrasive surface having a body and a plurality of abrasive elements positioned on the exterior face of the body. Each abrasive element is preferably pyramidal in shape. For ease of manufacture and durability, the abrasive elements and the body of the abrasive surface are both formed from a single piece of the same material. The abrasive surface is positioned above the top of the housing and is freely supported by the housing such that the abrasive surface can spin independently of the housing. The device further comprises a drive means for spinning the abrasive surface. The drive means mechanically connects the motor with the abrasive surface so that the motor, when activated, causes the abrasive surface to spin in a plane that is generally parallel to the plane of the floor upon which the skin exfoliating device is placed. The abrasive surface body preferably includes at least one concave portion and at least one convex potion so that the abrasive surface, when spinning, is compatible with the contours of the bottom of a person's foot as different portions of the bottom of the person's foot is moved over the abrasive surface to exfoliate skin. The device further comprises movement retardation means on the bottom surface of the base of the housing to help prevent the device from moving on the floor upon which it is placed. The movement retardation means allows the device to remain stationary when in use without requiring the device to be held in a hand.

A key object of the present invention is to provide a device to remove dead skin and calluses that may be operated in a wet environment like a shower. The device may be operated while the person is in the shower so that the person's dead skin and calluses are softened by the water to facilitate the removal of the dead skin and calluses. The device can also be used in a dry environment.

It is another object of the present invention to provide a battery-powered, motorized, and spinning device so that it is portable and may be used without requiring significant physical exertion or stamina.

It is another object of the present invention to provide a device which is operated in a hands-free manner so that a person does not have to hold the device while the device is being operated.

It is a further object of the present invention to provide a device with an abrasive surface that has both convex and concave portions so that as the abrasive surface spins, it is compatible with the contours of the bottom of the foot and is both comfortable and effective when being used to exfoliate the skin on the heel, ball, or arch of the foot.

It is also an object of the present invention to provide a device with an abrasive surface with a body and abrasive elements that are made from a single piece of the same material so that the abrasive surface may be easily and inexpensively manufactured using injection molding manufacturing methods. By manufacturing the abrasive surface body and abrasive elements as a single piece, there is no need to adhere or fasten abrasive elements to the abrasive surface body using glue or other fastening methods. This further reduces the cost of manufacturing the abrasive surface and also minimizes the breaking off or wearing away of abrasive elements with continued use which increases the durability of the abrasive surface.

It is additionally an object of the present invention for the device to have movement retardation means, such as suction cups or rubber pads, so that it can be placed on the floor of a shower so that the device will not move while it is being used to remove dead skin and calluses. In this way, a person will not slip or fall while using the device because it will remain stationary while the person moves the bottom of his or her foot across the device's abrasive surface.

These and other features, aspects, and advantages of the present invention will become better understood with regard to the following description, appended claims, and accompanying drawings.

### BRIEF DESCRIPTION OF THE DRAWINGS

Referring particularly to the drawings for the purpose of illustration only and not limitation, there is illustrated:

FIG. 1 is a perspective view of a preferred embodiment of the present invention;

FIG. 2 is a side plan view of the preferred embodiment of the present invention shown in FIG. 1 illustrating a foot shown in broken lines and positioned to enable the spinning abrasive surface to be used to remove dead skin and calluses from the bottom of the foot;

FIG. 3 is an enlarged view of the encircled portion of FIG. 2.

FIG. 4 is a top plan view of the preferred embodiment of the present invention shown in FIG. 1;

FIG. 5 is an enlarged view of the encircled portion of FIG. 3.

FIG. 6A is a cross-sectional view of the preferred embodiment of the present invention shown in FIG. 4 taken along the line 6A-6A;

FIG. 6B is a cross-sectional view of the preferred embodiment of the present invention shown in FIG. 4 taken along the line 6B-6B;

FIG. 7 is a bottom plan view of the preferred embodiment of the present invention shown in FIG. 1;

### DESCRIPTION

Although specific embodiments of the present invention will now be described with reference to the drawings, it should be understood that such embodiments are by way of example only and merely illustrative of but a small number of the many possible specific embodiments which can represent applications of the principles of the present invention. Various changes and modifications obvious to one skilled in the art to which the present invention pertains are deemed to be within the spirit, scope, and contemplation of the present invention as further defined in the appended claims.

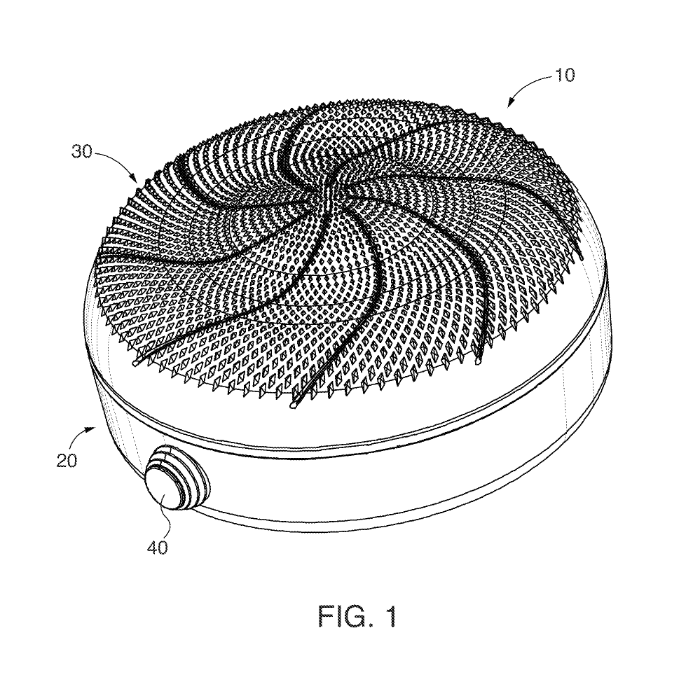

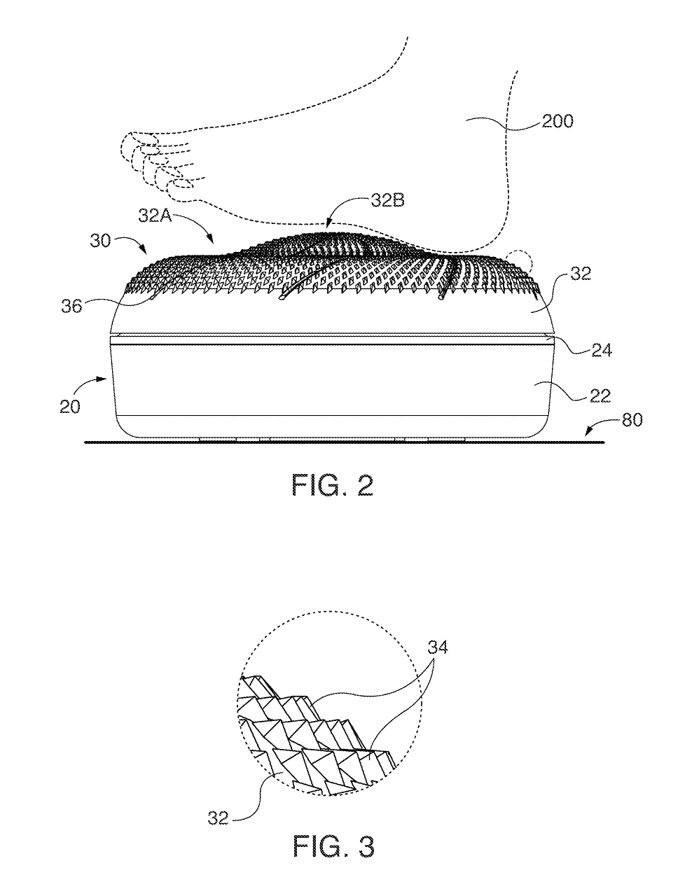

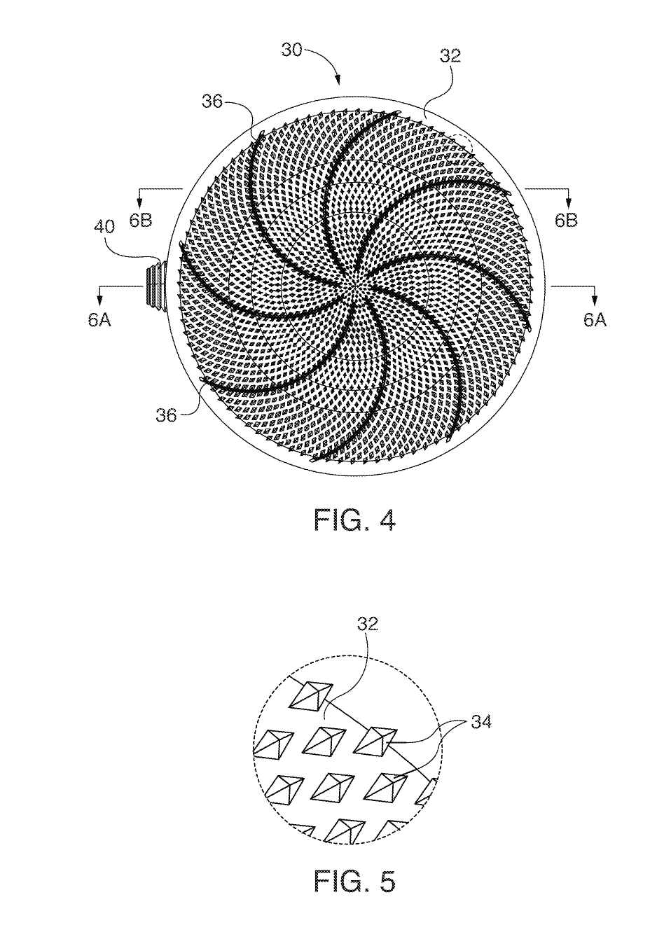

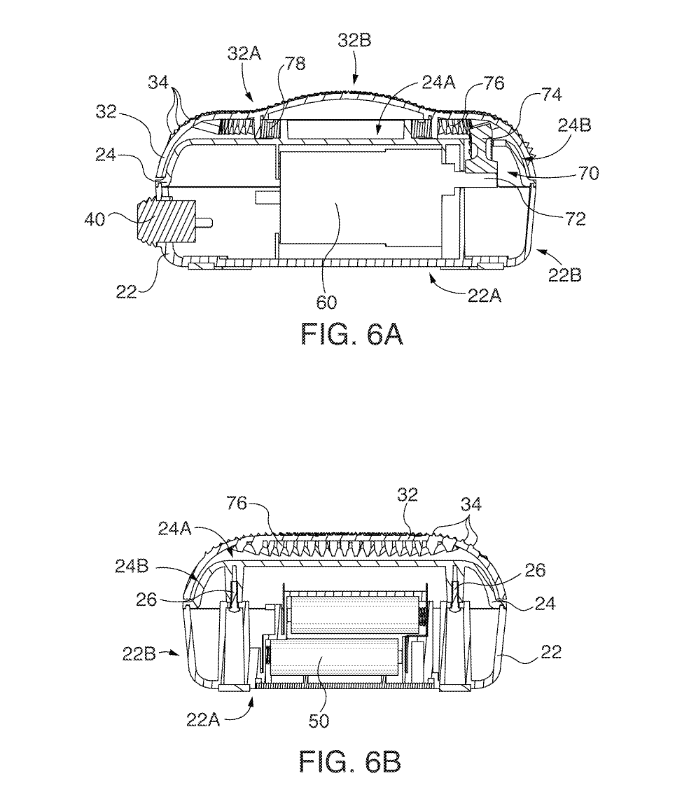

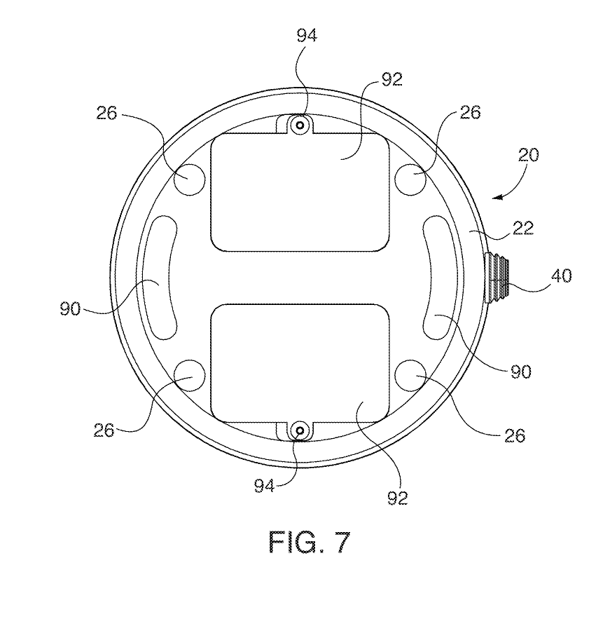

As shown in FIGS. 1 through 7, a preferred embodiment of an improved skin exfoliating device 10 comprises a generally cylindrical housing 20 and an abrasive surface 30 positioned above and coupled to the housing 20 such that the abrasive surface 30 can spin independently of the housing 20. The housing 20 comprises a base 22 and a top 24 connected to the base 22. The abrasive surface 30 comprises a body 32 and a plurality of abrasive elements 34 positioned on the exterior face of the body 32. An on-off switch 40 is positioned on the exterior of the side of the housing 20. A power source 50 is positioned within the housing 20. A motor 60 is positioned within the housing 20 and is electrically connected to the power source 50 and the on-off switch 40. A drive means 70 for spinning the abrasive surface 30 mechanically connects the motor 60 to the abrasive surface 30 so that the motor 60, when activated, causes the abrasive surface 30 to spin in a plane that is generally parallel to the plane of a floor 80 upon which the skin exfoliating device 10 is preferably placed during use. Movement retardation means such as rubber pads 90 are positioned on the exterior face of the base 22 to help prevent the skin exfoliating device 10 from moving on the floor 80 so that the skin exfoliating device 10 can be operated hands free.

A key innovation of the present invention is an abrasive surface 30 having a body 32 and a plurality of abrasive elements 34 positioned on the exterior face of the body 32 wherein each abrasive element 34 and the body 32 are together formed from a single piece of the same material. Suitable materials from which to form the abrasive surface include nylon, acrylonitrile butadiene styrene (ABS), acetal polyoxymethylene (POM) with 25% glass fiber, and other similarly hard materials which may be formed using injection molding or additive manufacturing methods, are resistant to damage or rusting from contact with water, and are safe for placing in contact with human skin.

Each abrasive element 34 is preferably generally pyramidal in shape, and most preferably, each abrasive element 34 is in the shape of a right pyramid having a base in the shape of a rhombus. Each abrasive element 34 is large enough so that the abrasive element 34 is effective at exfoliating skin and small enough so that the abrasive element 34 will not injure a person's foot 200 when the abrasive surface 30 is spinning and the person's foot 200 is placed in contact with the abrasive surface 30 during use. In an alternative embodiment, it is envisioned that the abrasive elements may be conical, cylindrical, thorn shaped, scale shaped, or another suitable shape.

Preferably, the body 32 includes at least one concave portion 32A and at least one convex portion 32B so that the abrasive surface 30, when spinning, is compatible with the contours of the bottom of a person's foot 200 as different portions of the bottom of the person's foot 200 is moved over the abrasive surface 30 to exfoliate skin. In an alternative embodiment, it is envisioned that the sizes and number of the concave and convex portions may be different.

Preferably, the body 32 includes at least one groove 36 that extends from approximately the center of the body 32 to the outer edge of the body 32. The groove 36 provides a channel through which water can flow to help prevent water from pooling on the face of the abrasive surface 30. Such pooling can reduce the effectiveness of the abrasive elements 34 at exfoliating skin when the device 10 is in use.

The housing 20 comprises a base 22 which has a generally horizontal bottom portion 22A and a generally vertical side portion 22B and a top 24 which has a generally horizontal top portion 24A and a generally vertical side portion 24B connected to the base 22 by one or more fasteners such as screws 26. In an alternative embodiment, it is envisioned that the base and the top are connected by other fasteners, such as adhesive. The on-off switch 40, power source 50, and motor 60 are electrically connected, such as by wires (not shown), so that when the on-off switch 40 is placed in the “on” state the motor 60 will be activated, and when the on-off switch 40 is placed in the “off” state the motor 60 will be deactivated. Preferably, the motor 60 is a DC motor, and the power source 50 is at least one battery so that the device 10 is portable and does not require AC power from a conventional wall outlet. However, it is envisioned that in alternative embodiments other suitable types of motors or power sources may be used. The power source 50 and motor 60 are positioned within the housing 20, and the housing 20 is preferably water resistant so that the power source 50, the motor 60, and the electrical connection between the on-off switch 40, the power source 50, and the motor 60 will not be damaged when the device 10 is used in a wet environment such as a shower.

The motor 60 and abrasive surface 30 are connected by a drive means 70 such that when the motor 60 is activated the motor 60 will cause the abrasive surface 30 to spin in a plane that is generally parallel to the plane of the floor 80 upon which the device is placed when in use. The drive means 70 for spinning the abrasive surface 30 is comprised of a shaft 72 extending from the motor 60, a bevel gear 74 located at the distal end of the shaft 72, and a crown gear 76 which is joined with the underside of the body 32 such that the teeth of the crown gear 76 are positioned near the outer edge of the body 32 and mesh with the teeth of the bevel gear 74. When the motor 60 is activated, the motor 60 causes the shaft 72 to spin which thereby causes the bevel gear 74 to spin which thereby causes the crown gear 76 to spin which thereby causes the body 32 to spin. A thrust bearing 78 couples the abrasive surface 30 to the housing 20 to provide support to the abrasive surface 30 and prevent the abrasive surface 30 from tilting when pressure is applied to the abrasive surface 30 by a person's foot 200 when the device 10 is in use. The thrust bearing 78 further allows the abrasive surface to spin freely when the device 10 is in use. In an alternative embodiment, it is envisioned that the drive means is comprised of a shaft which extends from the motor upward to the center of the body such that when the motor is activated the motor causes the shaft to spin which thereby causes the body to spin.

Preferably, the base 22 comprises one or more access doors 92 to facilitate gaining access to the power source 50. The access door 92 is fastened to the base 22 using one or more screws 94. In an alternative embodiment, it is envisioned that the access door is fastened to the base using magnets, tabs and slots, or other fastening means.

Movement retardation means such as textured rubber pads 90 are placed on the exterior surface of the base 22 to help prevent the device 10 from moving on the floor 80 upon which the device 10 is placed when in use. In an alternative embodiment, it is envisioned that the movement retardation means is one or more suction cups.

The device 10 is preferably placed on the floor 80 of a shower when it is to be used to exfoliate dead skin from the bottoms of a person's foot. The person preferably allows the dead skin to be softened by running water in the shower before using the device 10; however, the device 10 may also be used outside of a shower or on dry skin. When the on-off switch 40 is switched to the “on” state, the abrasive surface 30 will spin. The person places his/her foot 200 on to the spinning abrasive surface 30 and applies sufficient pressure with the foot 200 to allow the abrasive surface 30 to exfoliate the dead skin. The person may move his/her foot 200 across the abrasive surface 30 and place the curved portions of his/her foot 200 against the preferred one or more concave portions 32A or the preferred one or more convex portions 32B of the abrasive surface 30 to better exfoliate the skin on the curved portions of the foot 200.

The present invention is not intended to be restricted to any particular form or arrangement, or any specific embodiment, or any specific use, disclosed herein, since the same may be modified in various particulars or relations without departing from the spirit or scope of the claimed invention as shown and described herein. The preferred and alternative embodiments are intended only for illustration and disclosure of an operative embodiment and not to show all of the various forms or modifications in which this invention might be embodied or operated.

1. A skin exfoliating device comprising:
(a) a generally cylindrical housing comprising:
(i) a base; and
(ii) a top connected to the base;
(b) an on-off switch positioned on the exterior of the side of the housing;
(c) a power source positioned within the housing;
(d) a motor positioned within the housing and electrically connected to the power source and the on-off switch;
(e) an abrasive surface comprising:
(i) a body; and
(ii) a plurality of abrasive elements, wherein each abrasive element and the body are together formed from a single piece of the same material, the plurality of abrasive elements positioned on the exterior face of the body;
wherein the abrasive surface is positioned above the top of the housing and coupled to the housing such that the abrasive surface can spin independently of the housing;
(f) a drive means for spinning the abrasive surface, the drive means mechanically connecting the motor and the abrasive surface so that the motor, when activated, causes the abrasive surface to spin in a plane that is generally parallel to the plane of a floor upon which the skin exfoliating device is placed; and
(g) movement retardation means positioned on the exterior of the base of the housing to help prevent the skin exfoliating device from moving on the floor upon which it is placed so that it remains stationary when in use without requiring the skin exfoliating device to be held in a hand.

2. The skin exfoliating device according to claim 1 wherein the housing is water resistant.

3. The skin exfoliating device according to claim 1 wherein the base of the housing comprises an access door to facilitate gaining access to the power source.

4. The skin exfoliating device according to claim 1 wherein the power source is at least one battery.

5. The skin exfoliating device according to claim 1 wherein the abrasive surface body includes at least one concave portion and at least one convex portion so that the abrasive surface, when spinning, is compatible with the contours of the bottom of a person's foot as different portions of the bottom of the person's foot is moved over the abrasive surface to exfoliate skin.

6. The skin exfoliating device according to claim 1 wherein each abrasive element is generally pyramidal in shape. The skin exfoliating device according to claim 1 wherein the abrasive surface body includes at least one groove that extends from approximately the center of the abrasive surface body to the outer edge of the abrasive surface body.
